(19) United States
(12) Patent Application Publication
(10) Pub. No.: US 2025/0380872 A1
(43) Pub. Date: Dec. 18, 2025

(54) ULTRA-FINE 3D CAMERA DEVICE FOR PHOTOGRAPHING ROOT CANAL

(71) Applicant: BIGSCAN Co. Ltd., Seoul (KR)

(72) Inventor: Young Hoon SHIN, Seoul (KR)

(21) Appl. No.: 19/303,416

(22) Filed: Aug. 19, 2025

Related U.S. Application Data
(63) Continuation of application No. PCT/KR2024/010698, filed on Jul. 24, 2024.

(30) Foreign Application Priority Data
Aug. 7, 2023 (KR) 10-2023-0102685

(51) Int. Cl. A61B 5/00; H04N 23/55; H04N 23/67

(52) U.S. Cl. CPC A61B 5/0077; A61B 5/0088; H04N 23/55; H04N 23/672

(57) ABSTRACT

Provided is an ultra-fine 3D camera device, comprising: a probe including at least one optical fiber cable; a light source unit irradiating light of the same wavelength or different wavelengths onto a structure through the optical fiber cable; an optical lens unit detecting a reflected light signal irradiated onto the structure through the optical fiber cable, receiving the detected reflected light signal, and dispersing the light signal by color included in the light source unit; an image sensor unit converting the light signal formed by color through the optical lens unit into an electrical signal and outputting analog image data; and an image conversion processing unit converting the analog image data output from the image sensor unit into digital image data by color, performing signal processing on the converted digital image data by color, converting the converted digital image data into color image data, and outputting the color image data.

### FIELD

The present invention relates to an ultra-fine three-dimensional (3D) camera for endodontic photography, and more specifically, to a camera that receives incident waves 2 D images from a target object through an optical fiber at an imaging sensor to implement a 3D image.

### BACKGROUND

Endo Treatment is a field of dentistry that diagnoses and treats the pulp located deep inside the tooth. Root canal treatment is required when there is severe tooth decay, gum disease, or when the tooth's nerve tissue, the pulp, is infected by bacteria. During the treatment, the inflamed tissue is removed and the space is filled with a special filling material. The very narrow space called the root canal must be precisely located and the infected inflammation inside must be completely removed to prevent future problems. If this process is not completed sufficiently, secondary root canal treatment such as re-root treatment or root canal resection may be required.

Root canal treatment, which treats the delicate and complex structures called nerve canals, is a complex process that requires delicate yet high-level technical skills. During the process of opening the root canal (Access Opening), it is important to accurately find the canal orifice. This allows root canal treatment to begin after measuring the length of the nerve canal. For successful root canal treatment, it is important to quickly and accurately find the root canal entrance.

Traditionally, a small mirror is used to check the entrance to the root canal. The interior of the tooth reflected in the mirror is visually examined and the entrance to the root canal is found using a pile. In the case of traditional methods, it takes a lot of time and effort for the operator to find the entrance to the root canal because they rely on experience and manual dexterity. Recently, a microscope is also used to precisely examine the inside of the root canal. However, microscopes are very expensive and are a financial burden for use in private dental clinics. Therefore, a magnifying glass in the form of a glasses (Loupe) that allows only minimal magnification is mainly used. In the case of a microscope, 20× magnification is generally possible, but there are physical limitations in directly photographing the inside of the narrow and distant root canal. Because the inside of the root canal is indirectly enlarged and photographed through the periodontal lens, there are limitations to the image quality and the eye fatigue felt by the operator is also very high. In addition, it is not easy to find the root canal entrance because the distance from the microscope to the inside of the root canal is more than 60 cm, and the location of the root canal entrance varies depending on the tooth, and the structure is complex.

Since the image acquisition device containing electrical components in conventional oral cameras is directly inserted into the oral cavity, there is an electrical risk factor and the patient feels a strong foreign body sensation.

And since the image acquisition device containing electrical components of conventional oral cameras is directly inserted into the oral cavity, disposable sanitary plastic is used as a means of preventing cross-infection. However, since the intraoral camera is applied inside the mouth, sterilization rather than disinfection is absolutely necessary to prevent infection. To prevent infection, there is a high-pressure steam sterilization method that uses steam heated at high temperatures and pressures, but the material of conventional oral cameras cannot be sterilized using the high-pressure steam sterilization method, so there is a high risk of infection.

In addition, conventional oral cameras cover the image acquisition area with sanitary vinyl, which results in a deterioration in image quality.

### DETAILED DESCRIPTION

### Technical Problem

To solve the above-mentioned problems, the objective of the present invention is to provide an ultra-fine 3D camera device that can easily obtain 3D image data in a very narrow area such as the oral cavity using optical fiber.

Another objective of the present invention is to quickly and accurately detect the entrance of the pulp canal and the location of the pulp hole during root canal treatment.

Still another objective of the present invention is to resolve the problem of electrical risks by obtaining intraoral images using optical fibers that pose no electrical risks.

Still another objective of the present invention is to provide a detachable optical fiber probe to which high-pressure steam sterilization is applicable.

However, the technical tasks that this embodiment seeks to address are not limited to the technical tasks described above, and other technical tasks may exist.

### Technical Solution

According to an embodiment of the present invention for achieving the above-described objectives, an ultra-fine 3D camera device may comprise a probe including at least one optical fiber cable, a light source unit which irradiates light of the same wavelength or light of different wavelengths onto a structure through the at least one optical fiber cable, an optical lens unit which detects a irradiated onto the structure and reflected light signal through the at least one optical fiber cable, receives the detected reflected light signal, and disperses the light signal by colors included in the light source unit, an image sensor unit which converts the light signal formed by color through the optical lens unit into an electrical signal and outputs analog image data, and an image conversion processing unit which converts the analog image data output from the image sensor unit into digital image data by color, performs signal processing on the converted digital image data by color, performs signal processing and conversion of the converted digital image data by color into color image data, and outputs the color image data.

In an embodiment, the image sensor unit may be a phase detection image sensor or a quad phase detection image sensor.

In an embodiment, the image sensor unit obtains upper and lower phase difference information and left and right phase difference information from the at least one optical fiber cable using a phase detection function, calculates optical distance information of each image data using the obtained phase difference information, obtains color-specific imaging data including the calculated optical distance information, and can generate three-dimensional image data from the calculated optical distance information.

In an embodiment, the image conversion processing unit is included in the electical processing unit, and the electical processing unit may further comprise a control unit that transmits color image data output from the image conversion processing unit to a computing device via a communication unit, a power supply unit that supplies power to the ultra-fine 3D camera device, and a storage unit that stores digital image data.

In an embodiment, the at least one fiber optic cable extends through the body of the probe and protrudes from one end of the probe, wherein the probe can be made of any of the materials selected from stainless steel, titanium, tungsten carbide, nickel-chromium alloy, or a combination thereof for sterilization.

In an embodiment, the optical lens unit comprises a first lens, a second lens, an optical fiber bundle, a splitter, a protective glass film, and a lens unit case, and light irradiated from the light source unit passes through the optical fiber bundle, is split by the splitter, passes through the first lens, and is transmitted to the at least one optical fiber cable, and light reflected from the structure can be transmitted through the at least one optical fiber cable, passes through the first lens, the splitter, and the second lens, and then is transmitted to the image sensor.

According to another embodiment of the present invention, an ultra-fine 3D camera device comprises a probe, an optical lens unit, an electrical processing unit, and a battery unit, wherein the probe comprises at least one optical fiber cable, and the electrical processing unit comprises a light source unit that irradiates light of the same wavelength or light of different wavelengths to a structure through the at least one optical fiber cable, an image sensor that receives an optical signal, an electric circuit board that supports the light source unit and the image sensor and comprises a circuit that controls the ultra-fine 3D camera, an ON/OFF switch that supplies power, and a first signal connection terminal that receives power from a battery unit, and wherein the optical lens unit comprises an optical fiber bundle that receives and transmits light irradiated from the light source unit, a splitter that disperses light transmitted from the optical fiber bundle, a first lens that transmits light dispersed from the splitter to the probe, a second lens that receives light reflected after being irradiated to the structure and transmits it to an image sensor, a protective glass film, and a lens unit case, and wherein the battery unit comprises a second signal connection terminal that transmits power to the electrical processing unit, a battery cell and a charging terminal.

In an embodiment, the probe is detachably connected to the optical lens unit, the optical lens unit is detachably connected to the electrical processing unit, and the electrical processing unit and the battery unit are detachably connected to each other.

In an embodiment, the device further comprises a charger having a holder function, wherein the charger can wirelessly charge the ultra-fine 3D camera device.

In an embodiment, the image sensor unit may be a phase detection image sensor or a quad phase detection image sensor.

In an embodiment, the image sensor unit obtains upper and lower phase difference information and left and right phase difference information from the at least one optical fiber cable using a phase detection function, calculates optical distance information of each image data using the obtained phase difference information, obtains color-specific imaging data including the calculated optical distance information, and generates three-dimensional image data from the calculated optical distance information.

In an embodiment, the at least one fiber optic cable extends through the body of the probe and protrudes from one end of the probe, wherein the probe can be made of any material selected from stainless steel, titanium, tungsten carbide, nickel-chromium alloy, or a combination thereof for sterilization.

In an embodiment, the invention may provide a portable device which comprises an optical lens unit comprising an optical fiber bundle connected to the probe and receiving and transmitting light irradiated from a light source, a splitter that splits the light transmitted from the optical fiber bundle, a first lens that transmits the light split from the splitter to the probe, a second lens that receives light reflected after being irradiated on a structure and transmits it to an image sensor, a protective glass film, and a lens unit case, a probe coupler that is attached to one end of the probe and is connected to the optical lens unit, an image sensor that receives light from the optical lens unit, a light source and a display unit, and a case configured to accommodate the portable device, wherein the light source of the portable device may be arranged coaxially with the optical fiber bundle of the optical lens unit so as to irradiate light to the optical fiber bundle of the optical lens unit, and the image sensor may include a coupling unit that is arranged coaxially with the second lens so as to allow reflected light to be incident thereon.

In an embodiment, the light source can be visible light or invisible light.

In an embodiment, the image sensor may be any one of a 2D image sensor, a Phase Detection Autofocus (PDAF) image sensor and a Quad Phase Detection Autofocus (QPDAF) image sensor, and when the image sensor is a 2D image sensor, a 2D image can be displayed, and when the image sensor is a phase detection autofocus image sensor or a quad phase detection autofocus image sensor, a 3D image can be displayed.

### Advantageous Effects

According to the present invention, 3D image data of a very narrow area, such as the inside of the oral cavity, can be easily obtained using optical fiber.

In addition, according to the present invention, the entrance of the pulp canal and the location of the pulp hole can be quickly and accurately detected during root canal treatment.

In addition, according to the present invention, since intraoral images are acquired using optical fibers that pose no electrical risk, the problem of electrical risk can be addressed. In addition, according to the present invention, sterilization of the probe by high-pressure steam sterilization is possible by providing a detachable optical fiber probe.

### BRIEF DESCRIPTION OF THE DRAWINGS

FIG. 1 illustrates the configuration of an ultra-fine 3D camera device 100 according to an embodiment of the present invention.

FIG. 2 illustrated an ultra-fine 3D camera device 100 and a charger 200 according to an embodiment of the present invention.

FIG. 3 illustrates a portable video imaging device 300 according to an embodiment of the present invention.

FIG. 4 is a block diagram showing the internal configuration of an ultra-fine 3D camera 100 according to an embodiment of the present invention.

FIG. 5 is a block diagram showing the internal configuration of a computer device 500 according to an embodiment of the present invention.

### BEST MODE

According to an embodiment of the present invention, an ultra-fine 3D camera device may comprise a probe including at least one optical fiber cable, a light source unit which irradiates light of the same wavelength or light of different wavelengths onto a structure through the at least one optical fiber cable, an optical lens unit which detects a reflected light signal irradiated onto the structure through the at least one optical fiber cable and reflected, receives the detected reflected light signal, and disperses the light signal by color included in the light source unit, an image sensor unit which converts the light signal formed by color through the optical lens unit into an electrical signal and outputs analog image data, and an image conversion processing unit which converts the analog image data output from the image sensor unit into digital image data by color, performs signal processing on the converted digital image data by color, converts the converted digital image data by color into color image data, and outputs the converted digital image data by signal processing.

### BEST MODE FOR CARRYING OUT THE INVENTION

The embodiments of the present invention will be described in detail below with reference to the attached drawings, so that a person skilled in the art can easily implement the present invention. The present invention may have various modifications and embodiments, and specific embodiments are illustrated in the drawings and specifically described in the detailed description. However, this is not intended to limit the present invention to specific embodiments, but should be understood to include all modifications, equivalents, or substitutes included in the spirit and technical scope of the present invention.

In order to clearly explain the present invention, parts that are not related to the description are omitted from the drawings, and similar parts are given similar drawing reference numerals throughout the specification. In addition, when explaining with reference to drawings, even if the components are indicated by the same name, the drawing numbers may differ depending on the drawings, and the drawing numbers are described only for the convenience of explanation, and the concept, feature, function, or effect of each component is not limited and interpreted by the drawing numbers.

When describing each drawing, similar reference numerals are used to refer to similar components. The terms first, second, etc. may be used to describe various components, but such components should not be limited by such terms. The above terms are used solely to distinguish one component from another. For example, without departing from the scope of the present invention, the first component could be named the second component, and similarly, the second component could also be named the first component. The term “and/or” includes any combination of multiple related listed items or any one of multiple related listed items.

Unless otherwise defined, all terms used herein, including technical or scientific terms, have the same meaning as commonly understood by one of ordinary skill in the art to which this invention belongs.

Terms defined in commonly used dictionaries should be interpreted as having a meaning consistent with their meaning in the context of the relevant art, and should not be interpreted in an idealized or overly formal sense, unless expressly defined in this application.

Throughout the specification, when a part is said to be “connected” to another part, this includes not only the case where it is “directly connected” but also the case where it is “electrically connected” with another element in between. Additionally, when a part is said to “comprise” a component, this should be understood to mean that it may include other components, but not to the exclusion of other components, unless otherwise specifically stated, and does not preclude the presence or possibility of one or more other features, numbers, steps, operations, components, parts, or combinations thereof.

In this specification, a ‘part’ or ‘module’ includes a unit realized by hardware or software, and a unit realized using both. One unit may be realized using two or more pieces of hardware, and two or more units may be realized by one piece of hardware.

Hereinafter, an ultra-fine 3D camera device according to the present invention will be described in detail with reference to the attached drawings.

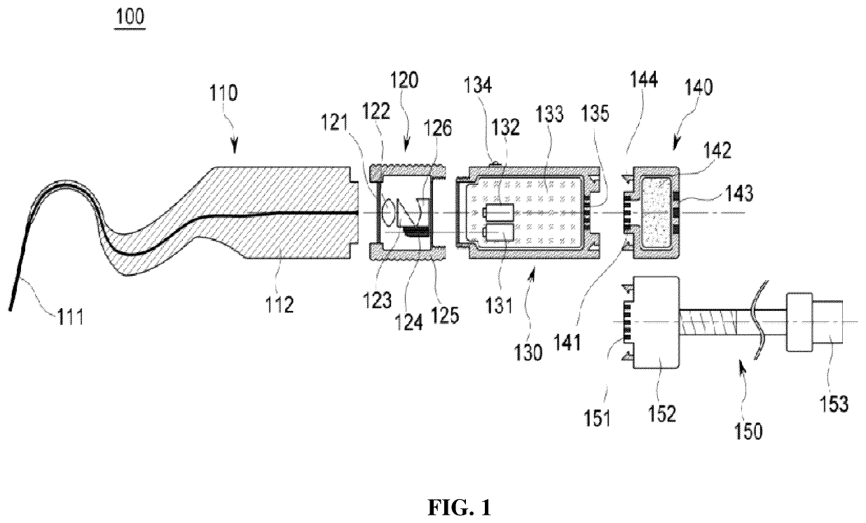

FIG. 1 illustrates an ultra-fine 3D camera device 100 according to an embodiment of the present invention.

Referring to FIG. 1, the ultra-fine 3D camera device 100 includes a probe 110 configured to allow an optical fiber cable 111 to pass through a probe member 112; an electrical processing unit 130 comprising a light source 131, an image sensor 132, an electrical circuit board 133, an ON/OFF switch 134, a first signal connection terminal 135; an optical lens unit 120 connected to the probe 110 and including a first lens 122, a second lens 126, a splitter 123, an optical fiber bundle 124, a protective glass film 121, and a lens unit case 125; and a battery unit 140 including a second signal connection terminal 141, a battery cell 142, and a charging terminal 143.

Specifically, the probe 110 is configured such that the optical fiber cable (111) passes through the probe member 112. Light irradiated from a light source 131 passes through an optical lens unit 120 and is then transmitted from one end of an optical fiber cable 111 to the other end. A portion of the optical fiber cable 111 protrudes from the end of the probe member 112. Measurement light can be irradiated to a target object through an optical fiber cable 111. The target object may be, for example, a dental structure in the oral cavity of a dental patient, but is not limited thereto, and may be structures used in various fields such as medicine, scientific research, industrial inspection, and security. The light reflected from the target object is re-injected into the optical fiber cable 111 and transmitted to the image sensor 132 through the optical lens unit 120. An optical lens (not shown) may be additionally coupled to one end of the optical fiber cable 111. The optical lens can be selected so that the light irradiated from the optical fiber cable 111 has an angle of view between 60 degrees and 150 degrees. However, the optical lens additionally coupled to the optical fiber cable 111 can be selected from various types according to the needs of a general technician, and the angle of view at this time can be selected from various types, such as 90 degrees, 60 degrees, or 120 degrees, etc. according to the design purpose.

The probe 110 may require exposure of the device to high pressure saturated steam at a temperature of typically about 121 degrees Celsius for a specified period of time to prevent infection and cross contamination. This process effectively kills bacteria, viruses, and other microorganisms, making the probe safe for use in medical procedures such as dental treatment procedures. For high-pressure steam sterilization, the material of the probe member 112 can be made of, for example, stainless steel, titanium, tungsten carbide, nickel-chromium alloy, or a combination thereof.

The electrical processing unit 130 comprises a light source 131, an image sensor 132, an electric circuit board 133, an ON/OFF switch 134, and a first signal connection terminal 135. The light source 131 can selectively use a multi-wavelength light source such as visible light or invisible light (NIR, SWIR). The choice of optical wavelength can be made in a variety of ways to obtain the desired image. For example, visible light has a wavelength of about 400 to 700 nanometers and is most commonly used as a light source in imaging devices. Near infrared (NIR) has a wavelength ranging from approximately 700 to 2500 nanometers. Near-infrared light can penetrate deeper into matter than visible light, making it useful for seeing through surface layers or examining the internal structure of an object. These properties are useful, for example, for visualizing blood vessels or tissues under the skin. Shortwave infrared (SWIR) has wavelengths ranging from about 1400 to 3000 nanometers. Imaging capabilities allow it to see through certain types of materials (such as silicon) that are impermeable to visible or near-infrared light, and can be used in low-light or dark environments.

In the present invention, various types of information can be obtained from a target object by selectively using a multi-wavelength light source such as visible light and invisible light. For example, some materials reflect certain wavelengths of light differently, which can provide important information about the materials' properties. In particular, it facilitates observation of inflammation, cracks, and other lesions inside the root canal that are difficult to identify with the naked eye.

The image sensor 132 may be a phase detection autofocus (PDAF) image sensor or a quad phase detection autofocus (QPDAF) image sensor. Phase-detection autofocus (PDAF) image sensors are an autofocus method that works by splitting incoming light into pairs of images and comparing them. Typically, a dedicated autofocus (AF) sensor is used separate from the image sensor. Once a pair of images are aligned, the camera considers them to be in correct focus. PDAF is generally faster and more reliable than contrast-detect autofocus, especially in bright situations.

Quad phase detection autofocus (QPDAF) image sensors divide each pixel into four subpixels, each of which can measure the direction of light. Using information about the direction of light, the camera can measure the distance between the subject and the lens and focus. The accuracy and speed of autofocus systems could potentially be improved greatly, as the system would have more data points to use when determining focus. QPDAF performs better in bright situations and provides faster and more accurate focusing than PDAF or Dual Pixel AF. Because each pixel in the QPDAF imaging sensor is divided into four sub-pixels, it can focus on any object, even if the object spans a wide range.

The image sensor 132 converts an optical signal formed by color through the optical lens unit 120 into an electrical signal and outputs analog image data. The image sensor (132) uses a phase detection function to obtain upper and lower and left and right phase difference information from an optical fiber cable (111), calculates optical distance information of image data for each structure (e.g., teeth) using the obtained phase difference information, obtains color-specific imaging data including the calculated optical distance information, and generates image data from the calculated optical distance information.

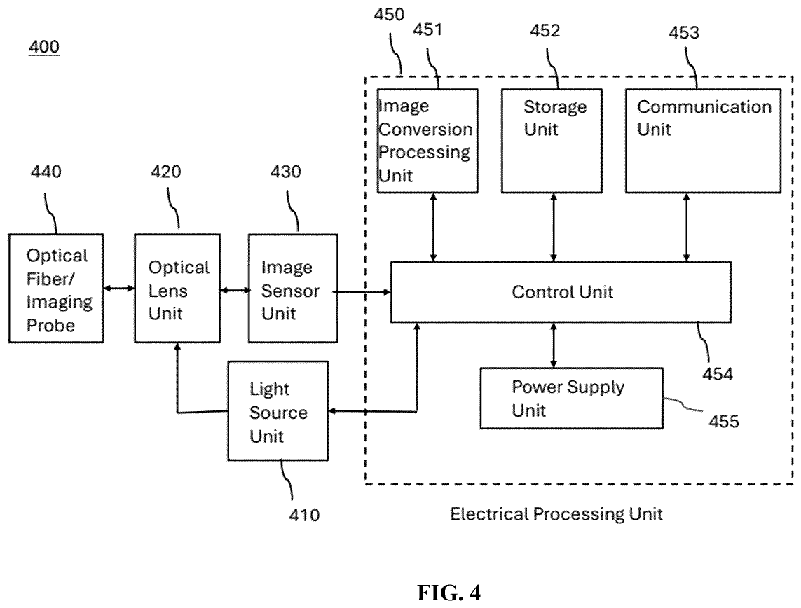

The circuit board 133 may include circuit devices for acquiring and processing image data. For example, it may include an image conversion processing unit 451, a storage unit 452, a communication unit 453, a control unit 454, a power supply unit 455, etc., as shown in FIG. 4 described later. A first signal connection terminal 135 is connected to one end of the circuit board 133, and the first signal connection terminal 135 can be connected to a second signal connection terminal 141 of the battery unit 140 to supply power. In order to turn the power of the electrical processing unit 130 ON/OFF, an ON/OFF switch 134 is electrically connected to the circuit board 133. The image conversion processing unit 451 converts analog image data output from the image sensor 132 into color-specific digital image data, and converts the converted color-specific digital image data into color image data through signal processing and outputs it. A detailed description of the image conversion processing unit 451 will be described later in connection with FIG. 4.

The optical lens unit 120 comprises a protective glass film 121, a first lens 122, a splitter 123, an optical fiber bundle 124, a lens unit case 125, and a second lens 126. The protective glass film 121 is intended to protect the internal components of the optical lens device from external factors such as dust, moisture, or physical damage. The surface of the protective glass film 121 may also have a coating applied to help reduce glare, reflections, and other optical aberrations. Light irradiated from a light source 131 is transmitted to an optical fiber bundle 124 of an optical lens unit 120. The optical fiber bundle 124 is connected to a splitter 123 and serves to divide light from a light source into multiple paths and transmit them. The first lens 122 serves to focus light. The second lens (126) may be a concave or aspherical lens.

The battery unit 140 comprises a second signal connection terminal 141, a battery cell (14), a charging terminal (14), and a connecting hook (14). The second signal connection terminal 141 serves as an interface that supplies power between the battery unit 140 and the electrical processing unit 130. It can also communicate information such as battery level, charging status, or other diagnostic data to the device's control system.

The battery cell 142 is the primary power source within the battery device. It stores electrical energy that can be used to power a 3D camera device. The battery cell 142 may be a single cell or a collection of smaller cells, depending on the power requirements of the 3D camera device. It can also be of different types such as lithium ion, nickel metal hydride, etc. depending on specific application and performance requirements.

The charging terminal 143 is an interface that supplies power when the battery cell is charged. While charging, it can be connected to an external power source, allowing electrical energy to flow into the battery cells. In FIG. 1, a charging method in which the 3D camera device is electrically coupled and connected is exemplified as a charging method, but both wired and wireless charging methods are possible, and the charging method may vary depending on the standard or specification.

The coupling hook 144 is part of the physical mechanism that secures the battery device. The coupling hook 144 ensures that the battery unit 140 is firmly attached to the electric circuit 130 in the correct position. Also, the battery unit 140 can be easily removed and replaced if necessary.

As shown in FIG. 1, a wired connection may be made using a cable 152 for wired connection. One end of the cable 152 for wired connection includes a signal connection terminal, and the other end includes a USB connection terminal 153. Image data acquired from an ultra-fine 3D camera device 100 can be transmitted to a computer device or portable device via a USB connection terminal 153.

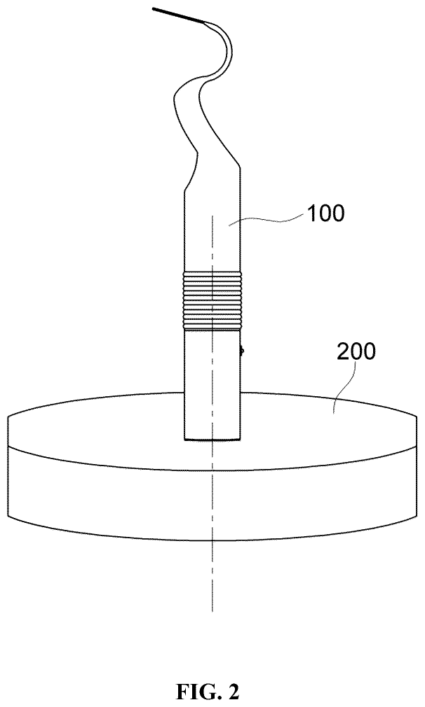

FIG. 2 is a drawing showing an ultra-fine 3D camera device 100 and a charger 200 according to an embodiment of the present invention.

Referring to FIG. 2, the ultra-fine 3D camera device 100 can be charged by being combined with a charger 200 having a holder function. The ultra-fine 3D camera device 100 may have a built-in connector or coupling mechanism that aligns with a corresponding connector of the charger 200. Depending on the design, this could include a physical plug and socket arrangement, magnetic connection, or even a wireless charging coil. When the ultra-fine 3D camera device 100 is placed into the charger 200, the connectors are aligned to create an electrical connection or electrical coupling between the ultra-fine 3D device 100 and the charger 200. Here, electrical connection or electrical coupling means wireless coupling, but is not limited thereto, and may also include wired connection. When the ultra-fine 3D camera device 100 and the charger 200 are electrically connected, the charging terminal 143 of the battery unit 140 is electrically connected to the corresponding terminal in the charger. This allows power to flow from the charger 200 to the battery cell 142 of the ultra-fine 3D camera device 100. The charger 200 is designed to control the charging speed so that the battery cell 142 is charged safely and efficiently. The charger 200 not only supplies power, but also serves as a holder for the ultra-fine 3D camera device 100. The design of the charger may include a space or slot to safely place the camera device while charging. Not only does this ensure that the charging connector is properly aligned, it also provides a convenient and secure way to store the device when not in use. The holder may be designed to hold the device in a specific orientation, such as vertical or sideways, depending on design requirements. The charger 200 includes various stabilizing features to protect the battery and the ultra-fine 3D camera device. For example, it may include overcharge protection to stop charging when the battery is fully charged, and thermal management to prevent overheating during charging.

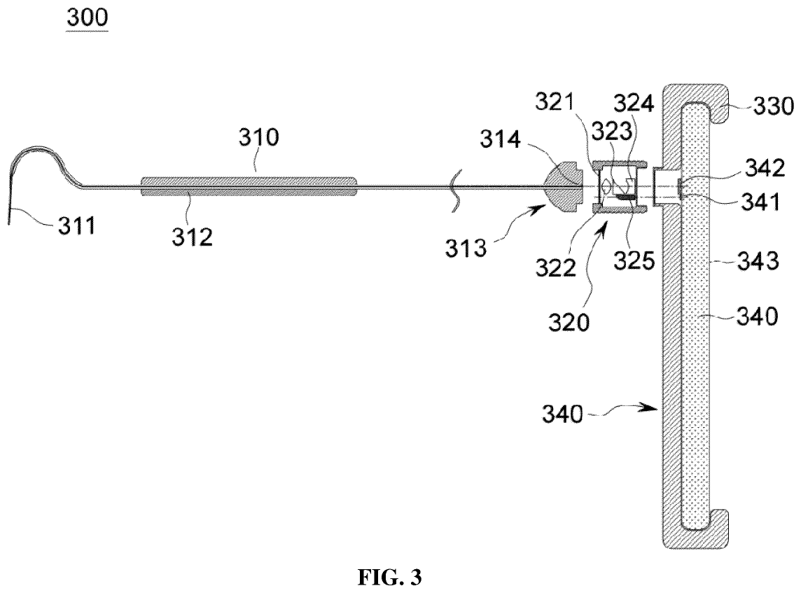

FIG. 3 illustrates a portable video imaging device 300 according to an embodiment of the present invention.

Referring to FIG. 3, the probe 310 comprises a fiber optic cable 311, a probe handle 312, a probe coupler 313, and a protective glass film 314. The optical lens unit 320 includes a protective glass film 321, a first lens 322, a splitter 323, a second lens 324, and an optical fiber bundle 325. A portable device case 330 is connected to an optical lens unit 320 and can accommodate a portable device 340. The portable device 340 comprises a light source 341, an image sensor 342, and a display unit 343.

Since the probe 310 and optical lens unit 320 perform the same functions as the probe 110 and optical lens unit 120 of FIG. 1, a detailed description will be omitted. The portable device 340 may be a portable electronic device such as a tablet PC, a mobile phone, a smart phone, an e-reader, a personal digital assistant (PDA), an enterprise digital assistant (EDA), a digital still camera, a digital video camera, a portable multimedia player (PMP), a personal navigation device (PND), a handheld game console, etc. The portable device 340 comprises a light source 341 and an image sensor 342.

In this embodiment, it is preferable that the light source 341 uses visible light, but a multi-wavelength light source such as invisible light (NIR, SWIR) may be optionally used. Light from a light source 341 of a portable device 340 passes through an optical lens unit 320 and is then irradiated to the structure through an optical fiber cable 311. Here, the structure may be tooth (teeth).

The optical lens unit (320) detects a reflected light signal reflected from a structure, receives the detected reflected light signal, and disperses the light signal into color-specific light signals included in the light source. In the image sensor (342), the optical signal formed by color through the optical lens unit is converted into an electrical signal and analog image data is output.

The image sensor 342 may use a 2D image sensor included in the portable device 340, but a phase detection auto-focus image sensor or a quad phase detection auto-focus image sensor may be used to obtain a 3D image. Image information input through a 2D image sensor is signal-processed by a processor (not shown) within a portable device 340 and displayed as an image through a display unit 343. When a phase detection auto-focus image sensor or a quad phase detection auto-focus image sensor is used to acquire a 3D image, the image sensor 342 acquires upper and lower and left and right phase difference information from the optical fiber cable 311 using the phase detection function, calculates optical distance information of image data for each structure using the acquired phase difference information, acquires color-specific imaging data including the calculated optical distance information, and generates image data from the calculated optical distance information.

A processor (not shown) included in a portable device 340 converts analog image data output from an image sensor 342 into color-specific digital image data, and performs signal processing on the converted color-specific digital image data to convert it into color image data and output it to a display unit 343. By combining a portable device 340 including a light source 341 and an image sensor 342 with an ultra-fine camera including an optical fiber cable, it has the advantage of being compact and easy to carry.

FIG. 4 is a block diagram showing the internal configuration of an ultra-fine 3D camera according to an embodiment of the present invention.

Referring to FIG. 4, the light source unit (410) selectively irradiates light of the same wavelength or light of different wavelengths to the structure through an optical fiber cable. Here, the structure can be a tooth (teeth) within the oral cavity, or it can be another structure depending on the application.

The optical lens unit 420 detects a reflected light signal that is irradiated and reflected on a structure through an optical fiber cable, receives the detected reflected light signal, and disperses the light signal into colors included in the light source unit 410.

The image sensor unit 430 converts an optical signal formed by color through the optical lens unit 420 into an electrical signal and outputs analog image data. The image sensor unit 430 may comprise a phase detection image sensor or a quad phase detection image sensor. The image sensor unit (430) uses a phase detection function to obtain upper and lower and left and right phase difference information from an optical fiber cable, calculates optical distance information of each tooth image data using the obtained phase difference information, obtains color-specific imaging data including the calculated optical distance information, and generates three-dimensional image data from the calculated optical distance information.

The electical processing unit 450 comprises an image conversion processing unit 451, a storage unit 452, a communication unit 453, a control unit 454, and a power supply unit 455.

The image conversion processing unit 451 converts analog image data output from the image sensor unit 430 into color-specific digital image data, and performs signal processing on the converted color-specific digital image data to convert it into color image data and output it.

The storage unit 452 is hardware that stores various data processed by the processor, and can store, for example, various programs or applications to be driven by the processor. The storage unit 452 may be implemented as a non-volatile memory such as ROM, PROM, EPROM, EEPROM, flash memory, PRAM, MRAM, RRAM, FRAM, etc., or may be implemented as a volatile memory such as DRAM, SRAM, SDRAM, PRAM, RRAM, FeRAM, etc. Additionally, the memory 110 may be implemented as an HDD, SSD, SD, Micro-SD, etc.

The communication unit 453 can connect to a network and receive content from an external device (not shown). The communication unit 453 may comprise a combination of modules including at least one of a wireless LAN, a Bluetooth communication unit, and a wired Ethernet. Additionally, the communication unit 1550 can receive a control signal from an external device. The control signal can be implemented as a Bluetooth type, an RF signal type, or a Wi-Fi type.

The control unit 454 transmits color image data output from the image conversion processing unit 451 to a computing device via the communication unit 453. The control unit 454 may include a processor, and the processor may be implemented as a CPU (central processing unit), a GPU (graphics processing unit), an AP (application processor), or an NPU (neural processing unit).

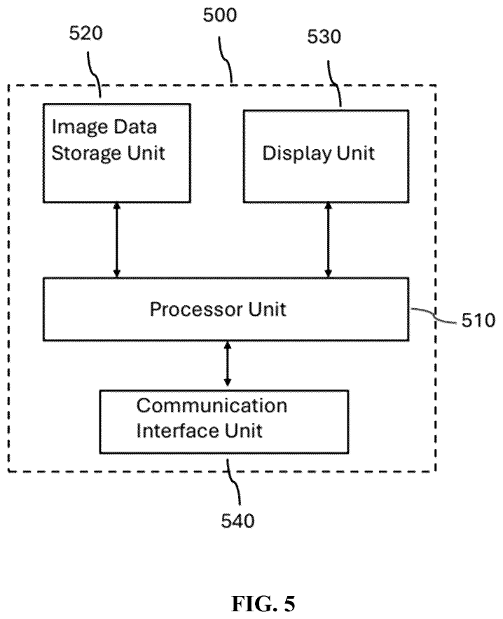

FIG. 5 is a block diagram showing the internal configuration of a computer device 500 according to an embodiment of the present invention.

The computer device 500 of FIG. 5 is a device that receives image data from an ultra-fine 3D camera, processes the data, and displays the image to the user through a display unit 520. Specifically, the computer device 500 comprises a processor unit 510, an image data storage unit 520, a display unit 530, and a communication interface unit 540.

The processor unit 510 can execute commands or at least one program for processing image data stored in the image data storage unit 520. The processor unit 510 can also perform overall functions for controlling a server (not shown) and process various operations within the server (not shown). The processor unit 510 can be implemented as an array of multiple logic gates or a general-purpose microprocessor. The processor unit 510 may be composed of a single processor or multiple processors. The processor unit 510 may be at least one of a CPU, GPU, and AP provided in the device 100, but this is only an example, and the processor 120 may be implemented in various other forms.

Image data transmitted through the communication unit 453 of the ultra-fine 3D camera can be received through the communication interface unit 540. The communication interface unit 540 can be connected to the communication unit 453 of the ultra-fine 3D camera via wired or wireless means, or can be connected to a network such as a local area network (LAN) and a wide area network (WAN). Additionally, it may be possible to connect to a dedicated channel for one-to-one communication with the user or the user's terminal.

The image data storage unit 520 stores image data transmitted from an ultra-fine 3D camera. The image data storage unit 520 may be implemented as a non-volatile memory such as ROM, PROM, EPROM, EEPROM, flash memory, PRAM, MRAM, RRAM, FRAM, etc., or may be implemented as a volatile memory such as DRAM, SRAM, SDRAM, PRAM, RRAM, FeRAM, etc. Additionally, the memory 110 may be implemented as an HDD, SSD, SD, Micro-SD, etc.

The device described above may be implemented as hardware components, software components, and/or a combination of hardware components and software components. For example, the devices and components described in the embodiments can be implemented using one or more general-purpose computers or special-purpose computers, such as a processor, a controller, an arithmetic logic unit (ALU), a digital signal processor, a microcomputer, a field programmable gate array (FPGA), a programmable logic unit (PLU), a microprocessor, or any other device capable of executing instructions and responding. A processing device can execute an operating system (OS) and one or more software applications running on the operating system. Additionally, the processing device may access, store, manipulate, process, and generate data in response to the execution of the software. For ease of understanding, the processing unit is sometimes described as being used alone, however, those skilled in the art will recognize that the processing unit may include multiple processing elements and/or multiple types of processing elements. For example, a processing unit may include multiple processors or one processor and one controller. Additionally, other processing configurations, such as parallel processors, are also possible.

Although the embodiments have been described above by way of limited examples and drawings, those skilled in the art will appreciate that various modifications and variations can be made from the above description. For example, suitable results may be achieved if the described techniques are performed in a different order than described, and/or if components of the described systems, structures, devices, circuits, etc. are combined or combined in a different manner than described, or if they are replaced or substituted by other components or equivalents.

Therefore, other implementations, other embodiments, and equivalents to the claims are also included in the scope of the claims described below.

Reference Numerals

| | | |
|---|---|---|
| 100: Ultra-fine 3D camera device | 110, 310: Probe | |
| 111, 311: Optical fiber cable | 112: Probe member | |
| 120, 320: Optical lens part | 121, 314: Protective glass film | |
| 122, 322: First lens | 123, 323: Splitter | |
| 124, 325: Optical fiber bundle | 125: Lens part case | |
| 126, 324: Second lens | 130: Electrical Processing Unit | |
| 131, 341: Light source | 132, 342: Image sensor | |
| 133: Circuit board | 134: ON/OFF switch | |
| 135, 141, 151: Signal connection terminal | 140: Battery part | |
| 142: Battery cell | 143: Charging terminal | |
| 144: Joining hook | 150: Wired connection part | |
| 152: Cable for wired connection | 153: USB connection terminal | |
| 200: Charger | 300: Micro camera combined with portable device | |
| 312: Probe handle | 313: Probe coupler | 330: Case 3 |
| 40: Portable device | 343: Display unit | |

1. An ultra-fine 3D camera device, comprising:
a probe including at least one optical fiber cable;
a light source unit that irradiates light of the same wavelength or light of different wavelengths to a structure through the at least one optical fiber cable;
an optical lens unit that detects a reflected light signal irradiated to the structure through the at least one optical fiber cable and reflected, and receives the detected reflected light signal and splits the light signal into colors included in the light source unit;
an image sensor unit that converts the light signal formed by color through the optical lens unit into an electrical signal and outputs analog image data; and
an image conversion processing unit that converts the analog image data output from the image sensor unit into color-specific digital image data, and converts the converted color-specific digital image data into color image data by signal processing and outputs the color image data.

2. The ultra-fine 3D camera device of claim 1,
wherein the image sensor unit is a phase detection image sensor or a quad phase detection image sensor.

3. The ultra-fine 3D camera device of claim 2,
wherein the image sensor unit obtains phase difference information of the upper and lower phase difference information and the left and right phase difference information from the at least one optical fiber cable using a phase detection function, calculates optical distance information of each image data using the obtained phase difference information, obtains color-specific imaging data including the calculated optical distance information, and generates 3D image data from the calculated optical distance information.

4. The ultra-fine 3D camera device of claim 1,
wherein the image conversion processing unit is included in a electrical processing unit, and the electrical processing unit further includes a control unit that transmits color image data output from the image conversion processing unit to a computing device through a communication unit, a power supply unit that supplies power to the ultra-fine 3D camera device, and a storage unit that stores digital image data.

5. The ultra-fine 3D camera device of claim 1,
wherein the optical fiber cable passes through the main body of the probe and protrudes from one end of the probe, and the probe is made of any material selected from stainless steel, titanium, tungsten carbide, nickel-chromium alloy, or a combination thereof for sterilization.

6. The ultra-fine 3D camera device of claim 1,
wherein the optical lens unit comprises a first lens, a second lens, an optical fiber bundle, a splitter, a protective glass film, and a lens unit case,
wherein light irradiated from the light source unit passes through the optical fiber bundle, is split by the splitter, passes through the first lens, and is transmitted to the optical fiber cable,
and light reflected from the structure is transmitted through the at least one optical fiber cable, passes through the first lens, the splitter, and the second lens, and is then transmitted to the image sensor.

7. An ultra-fine 3D camera device comprising a probe, an optical lens unit, an electrical processing unit, and a battery unit,
wherein the probe comprises at least one optical fiber cable,
the electrical processing unit comprises a light source unit that irradiates light of the same wavelength or light of different wavelengths to a structure through the at least one optical fiber cable, an image sensor that receives an optical signal, an electric circuit board that supports the light source unit and the image sensor and includes a circuit that controls the ultra-fine 3D camera, an ON/OFF switch that supplies power, and a first signal connection terminal that receives power from a battery unit,
the optical lens unit comprises an optical fiber bundle that receives and transmits light irradiated from the light source unit, a splitter that disperses light transmitted from the optical fiber bundle, a first lens that transmits light dispersed from the splitter to the probe, a second lens that receives light reflected after being irradiated to the structure and transmits it to an image sensor, a protective glass film, and a lens unit case, and
the battery unit comprises a second signal connection terminal that transmits power to the electrical processing unit, a battery cell, and a charging terminal.

8. The ultra-fine 3D camera device of claim 7,
wherein the probe is detachably connected to the optical lens unit, the optical lens unit is detachably connected to the electrical processing unit, and the electrical processing unit and the battery unit are detachably connected to each other.

9. The ultra-fine 3D camera device of claim 7,
further comprising a charger having a holder function, wherein the charger wirelessly charges power with the ultra-fine 3D camera device.

10. The ultra-fine 3D camera device of claim 7,
the image sensor unit is a phase detection image sensor or a quad phase detection image sensor.

11. The ultra-fine 3D camera device of claim 10,
wherein the image sensor unit obtains phase difference information of the upper and lower phase difference information and the left and right phase difference information from the at least one optical fiber cable using a phase detection function, calculates optical distance information of each image data using the obtained phase difference information, obtains color-specific imaging data including the calculated optical distance information, and generates 3D image data from the calculated optical distance information.

12. The ultra-fine 3D camera device of claim 7,
wherein the at least one optical fiber cable passes through the main body of the probe and protrudes from one end of the probe, and the probe is made of any material selected from stainless steel, titanium, tungsten carbide, nickel-chromium alloy, or a combination thereof for sterilization.
